(19) United States
(12) Patent Application Publication
(10) Pub. No.: US 2005/0283120 A1
(43) Pub. Date: Dec. 22, 2005

(54) Adapable safety syringe

(76) Inventor: Hsien-Tsung Wang, Taipei (TW)

Correspondence Address: Hsien Tsung WANG, 4Fl. No. 27, Lane 160, Hsin Sheng S. Rd., Sec. 1, Taipei, 100, TW

(21) Appl. No.: 10/870,979

(22) Filed: Jun. 21, 2004

(51) Int. Cl. A61M 5/32

(52) U.S. Cl. 604/198

(57) ABSTRACT

A safety syringe comprises a plunger, a hollow cylindrical, staged adapter fitted on a forward nose of the plunger, a projection on a circumference of the adapter, a hypodermic needle mounted on a forward end of the adapter, and a sheath for enclosing the adapter and the needle, the sheath comprising a T-shaped groove having a left recess with the projection disposed therein in a non-operating position, a right slot, and an intermediate cross piece in a horizontal portion thereof. The projection slides along the groove for causing the needle to dispense fluid through a forward aperture of the sheath. Also, responsive to again disposing the projection in the recess after dispensing fluid, rotate the adapter to move the projection from the recess to and lock in the slot by passing under the cross piece. The sheath is adapted to couple to any typical syringe plunger or IV infusion set.

### BACKGROUND OF THE INVENTION

1. Field of the Invention

The present invention relates to hypodermic needles and more particularly to an improved safety syringe being highly adaptable to one of a variety of syringe plungers or intravenous (IV) adapters.

2. Description of Related Art

There is no hypodermic needle currently on the market that completely and satisfactorily solves the safety problem from accidentally pricking after its use and thereby contaminating the medical worker with microorganisms or blood on the needle. For example, U.S. Pat. No. 6,093,170 disclosed a structure safety syringe comprising a barrel 10, a plunger 20, and a sleeve 30 in which the barrel 10 and the plunger 20 are well known devices. The patent is characterized in that a tab 13 is disposed near a hypodermic needle 12 on a circumferential surface of the barrel 10, a U-shaped guide slot 33 is disposed lengthwise along a wall of the sleeve 30, and an insertion section 35 aligned with the tab 13 is formed at interior ends of the guide slot 33. However, the sleeve 30 is not safe in use due to the following reasons: The sleeve 30 is made of plastic by injection molding. The insertion section 35 is not able to seal the tab 13 completely because the insertion section 35 has an inherent flexibility of the plastic. Hence, the tab 13 can be advanced again for at least a second dispensing of fluid after its use. As a result, the claimed safety feature is compromised. For accommodating the barrel 10 and the sleeve 30 and facilitating a smooth sliding of the tab 13 along the guide slot 33, a size of the barrel 10 is required to modify for overcoming the adverse affection caused by provision of the tab 13. As such, the barrel 10 is not readily adapted to the conventional international specification. However, the modification of the barrel 10 is costly due to a high price molding. In addition, a compatibility of the barrel 10 with other similar products is very low. That is, the barrel 10 cannot be used as a replacement of any of other commercially available syringe barrels. Additionally, in response to the modification of the barrel 10, issues such as an acceptable smoothness of the barrel 10 sliding in the plunger 20, tightness, and safety have not be satisfactorily solved by the patent. Hence, a need for improvement exists.

### SUMMARY OF THE INVENTION

It is an object of the present invention to provide a highly adaptable safety syringe in which a retractable needle retracts within a sheath and locks in a lock slot of the sheath immediately after fluid dispensing so that a second advancing of the contaminated needle for dispensing fluid again is prohibited. By utilizing this syringe, safety in dispensing fluid by a medical worker is greatly increased.

It is another object of the present invention to provide a highly adaptable safety syringe in which the sheath is adapted to couple to one of a variety of typical syringe plungers or IV infusion sets by means of the adapter without modifying the plunger or the IV infusion set, thereby greatly increasing compatibility and adaptability of the invention.

It is a further object of the present invention to provide a highly adaptable safety syringe comprising a plunger, a hollow cylindrical, staged adapter fitted on a forward nose of the plunger, a projection on a circumference of the adapter, a hypodermic needle mounted on a forward end of the adapter, and a sheath for enclosing the adapter and the needle, the sheath comprising a T-shaped groove so as to define a sliding of the projection therealong and cause the needle to extend through a forward aperture of the sheath for dispensing fluid or lock the projection therein.

The above and other objects, features and advantages of the present invention will become apparent from the following detailed description taken with the accompanying drawings.

### BRIEF DESCRIPTION OF THE DRAWINGS

FIG. 1 is an exploded perspective view of a first preferred embodiment of adaptable safety syringe according to the invention;

FIG. 1A is a perspective view of projection shown in FIG. 1;

FIG. 2 is a partial cross-sectional view of the assembled syringe in a non-operating position;

FIG. 2A is a greatly enlarged, fragmentary, cross-sectional view shown in a circle of FIG. 2;

FIG. 3 is a view similar to FIG. 2 where the syringe is in an operating position;

FIGS. 4, 5, and 6 are cross-sectional views of a portion of the syringe for illustrating three operating positions of the projection respectively;

FIG. 7 is a cross-sectional view taken along line 7-7 of FIG. 5;

FIG. 8 is a cross-sectional view taken along line 8-8 of FIG. 6;

FIG. 9 is a perspective view of a second preferred embodiment of adaptable safety syringe according to the invention;

FIG. 10 is a perspective view of a third preferred embodiment of adaptable safety syringe according to the invention; and

FIG. 11 is a perspective view of a fourth preferred embodiment of adaptable safety syringe according to the invention.

### DETAILED DESCRIPTION OF THE PREFERRED EMBODIMENTS

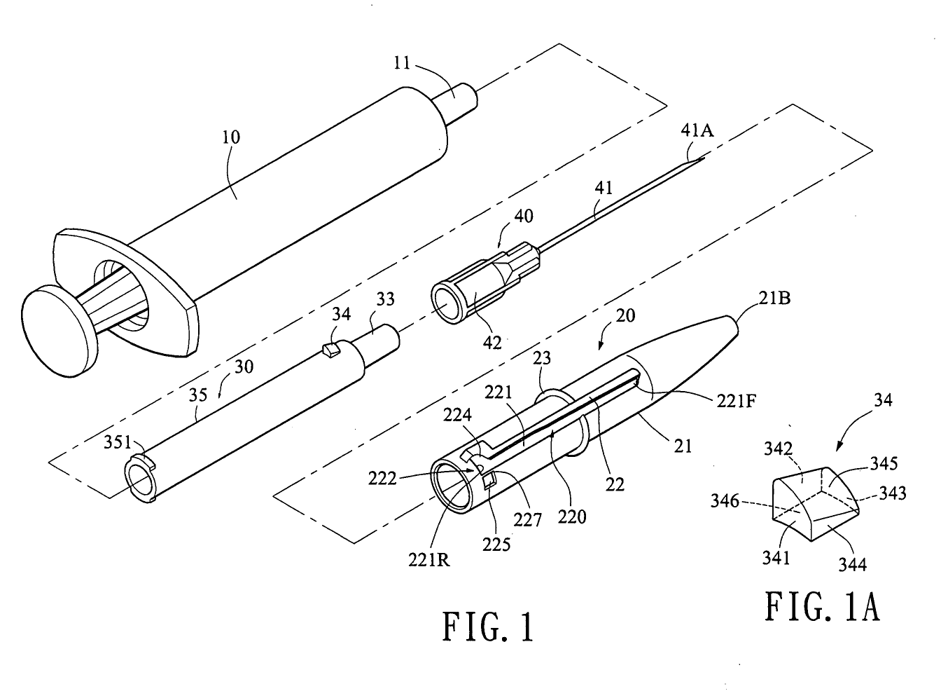

Referring to FIGS. 1, 1A, 2, 2A, and 3, there is shown a highly adaptable safety syringe constructed in accordance with a first preferred embodiment of the invention. The syringe comprises a plunger 10; a hollow cylindrical adapter 30 snugly fitted on a forward nose 11 of the plunger 10 in this embodiment, the adapter 30 including two opposite rear flanges 351 on a circumference thereof, a rear first cylindrical section 35, a forward second cylindrical section 33 having an outer diameter smaller than that of the first cylindrical section 35, and a six-sided projection 34 formed on the circumference of the first cylindrical section 35 adjacent the second cylindrical section 33; a hypodermic needle unit 40 including a needle 41 having a pointed end 41A and a needle housing 42 snugly fitted on the second cylindrical section 33; and a sheath 20 for enclosing the projection 34 and the needle unit 40 in a non-operating position.

The projection 34 is shaped as one having a lower forward side, a higher rearward side, a lower right side, a higher left side, and a section of D. As such, the projection 34 is facilitated to fit in the sheath 20 and lock in a lock slot 225 of the sheath 20. In detail, the projection 34 comprises a first side of D shape 341, a second side of trapezoidal shape 342, a third side of D shape 343, a fourth side of trapezoidal shape 344, a square top 345, and a square, flat bottom 346. The area of the first side 341 is larger than that of the second side 342. The area of the second side 342 is larger than that of the third side 343. The area of the third side 343 is larger than that of the fourth side 344. The top 345 is arcuate downward from the higher second side 342 to the lower fourth side 344. Also, the top 345 is arcuate downward from the higher first side 341 to the lower third side 343. Such projection 34 is thus facilitated to slide to lock in the lock slot 225 as detailed later.

As shown in FIG. 1, the sheath 20 comprises a hollow body 21, a lengthwise T-shaped groove 220 on the body 21, a dust cover 22 formed to cover the groove 220 for hygienic purpose, and an aperture 21B in a forward end of the body 21 for permitting the needle 41 to extend or retract therethrough. A length of the groove 220 is smaller than that of the body 21. The groove 220 comprises a vertical portion 221 including a forward stop 221F and a rearward intersection 221R and a horizontal portion 222 including a first (left) square recess 224, a second (right) square lock slot 225, and an intermediate cross piece 227 abutted on the lock slot 225.

In assembly, fit the adapter 30 on the forward nose 11 of the plunger 10. Next, fit the needle housing 42 on the second cylindrical section 33. Finally, put the sheath 20 on the adapter 30 for covering the needle unit 40 and the projection 34 which is also defined in the groove 220. As such, the needle 41 is allowed to extend or retract through the sheath 20.

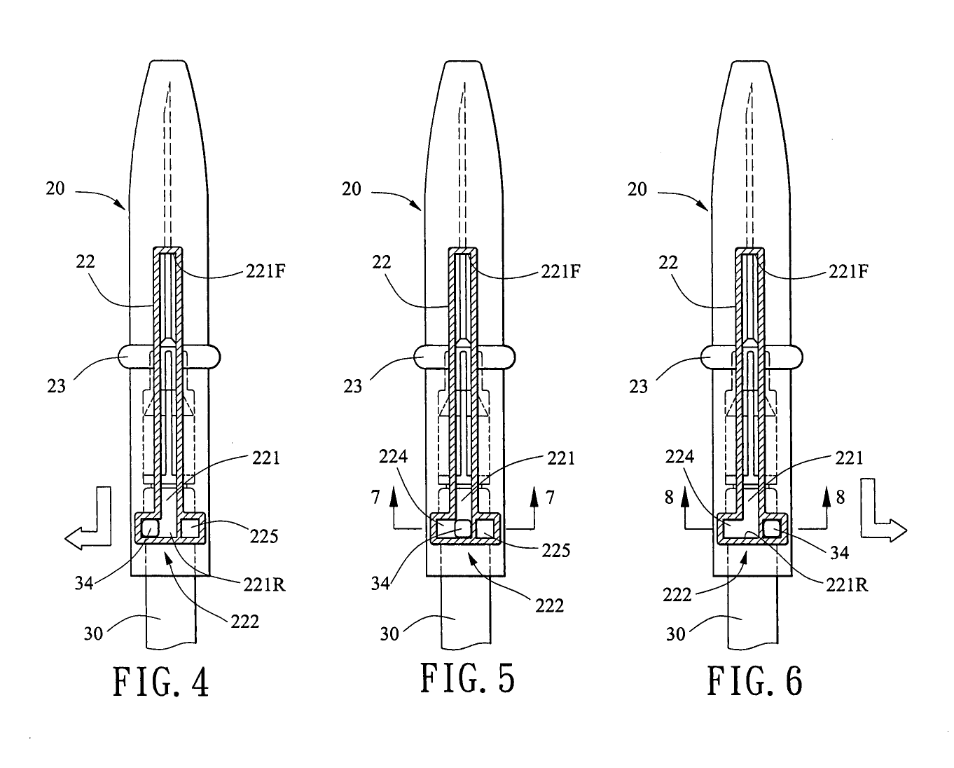

Referring to FIGS. 4, 5, 6, 7, and 8, the projection 34 is located at the recess 224 prior to use (see FIG. 4). As such, the needle unit 40 is sealed by the sheath 20 with the needle 41 being protected by the sheath 20. This is a safe configuration. For dispensing fluid, move the projection 34 from the recess 224 to the groove 220 via the horizontal portion 222 and the rearward intersection 221R. Next, push the plunger 10 forward to cause the projection 34 to slide along the groove 220 and the needle 41 to extend through the aperture 21B until the projection 34 is stopped by the forward stop 221F. At this position, the needle 41 is completely exposed (see FIG. 3). The hand of a medical worker holding the sheath 20 is behind the needle 41 while dispensing fluid. As such, there is no need to worry that the hand of the medical worker is pricked accidentally by the needle 41 or the medical worker is contaminated by microorganisms or blood on the needle 41. Thus, the syringe of the invention is highly safe in use.

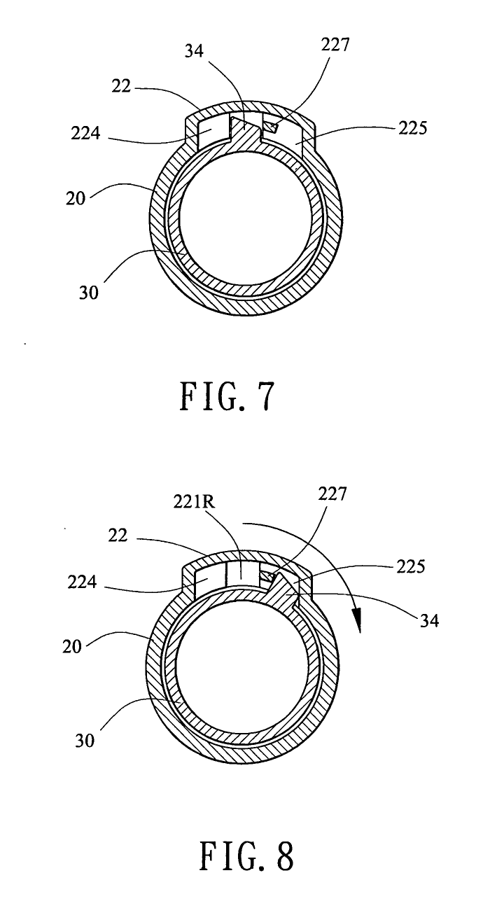

After fluid dispensing, simply slide the projection 34 rearward along the groove 220 from the forward stop 221F to the recess 224 via the rearward intersection 221R and the horizontal portion 222. At this position, the needle unit 40 is again concealed in the sheath 20. A clockwise rotation of the adapter 30 with respect to the sheath 20 will cause the projection 34 to move from the recess 224 (FIGS. 4 and 7) to the lock slot 225 (FIGS. 6 and 8). Also, the projection 34 is locked in the lock slot 225 once enters into the lock slot 225. At this position, the projection 34 is substantially prevented from disengaging the lock slot 225 accidentally since a left side of the top 345 of the projection 34 is much higher than a top surface of the abutted cross piece 227. As such, the needle 41 is always concealed by the sheath 20 at this position. Also, the hand of a medical worker holding the sheath 20 is behind the needle 41 while rotating the adapter 30. As such, there is no need to worry that the hand of the medical worker is pricked accidentally by the needle 41 or the medical worker is contaminated by microorganisms or blood on the needle 41. Thus, the syringe is highly safe.

One purpose of the syringe of the invention is to prevent a second advancing of the once contaminated needle 41 from dispensing fluid again after its use. For this reason, the recess 224 of the left vertical portion 221 of the T-shaped groove 220 is shaped to conform with the projection 34 and the right horizontal portion 222 thereof is formed as the intermediate cross piece 227 and the lock slot 225 which is also shaped to conform with the projection 34. The sheath 20 is formed of plastic by plastic molding. As such, the elongate cross piece 227 has a good flexibility. Further, the cross piece 227 is formed integrally with the sheath 20. The cross piece 227 can maintain its strength during and after the process of moving the projection 34 into or away from the lock slot 225 by passing the projection 34 under the cross piece 227. Moreover, the arcuate top 345 of the projection 34 can facilitate the passing of the projection 34 under the cross piece 227. Note that once the projection 34 is locked in the lock slot 225 with the needle 41 concealed in the sheath 20 after dispensing fluid the needle 41 cannot be used again. This can completely prevent criminals from using the syringe again by simply cleaning the used needle.

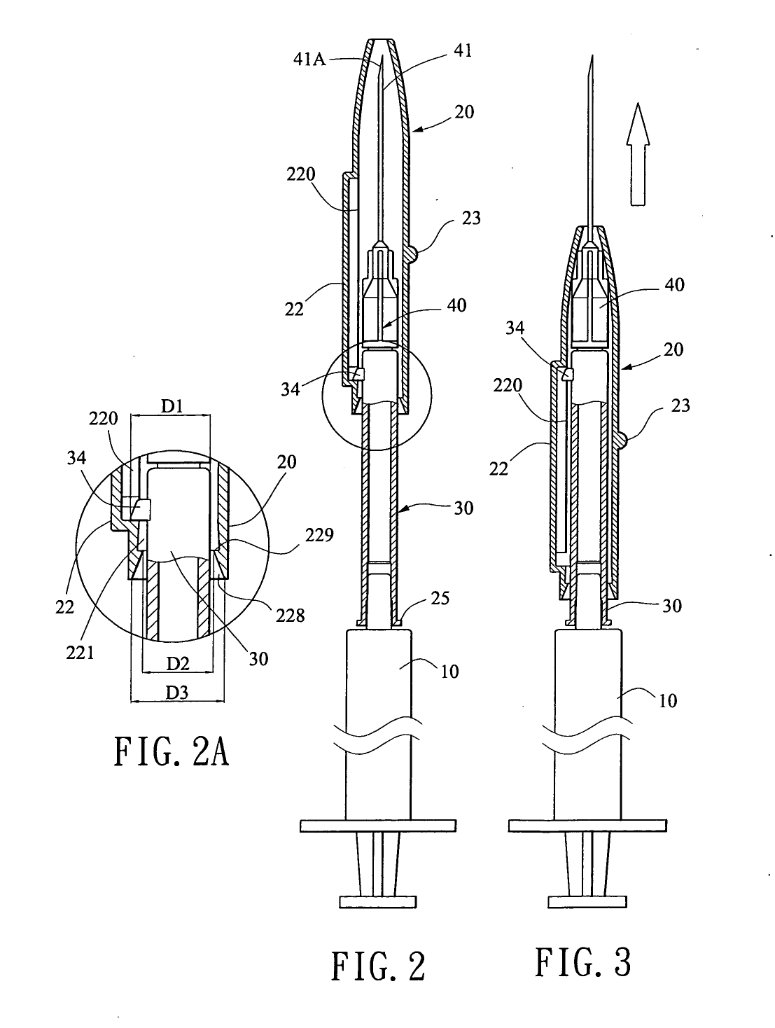

Referring to FIG. 1 again, a slip resistant ring 23 is formed on a circumference of the sheath 20 for facilitating a holding of the sheath 20 by hand Referring to FIG. 2A again, a bell shaped opening 228 and an abutted shoulder 229 are formed at a rear end of the sheath 20 in which the shoulder 229 has an inner diameter smaller than that of the sheath 20 at the rear end of the sheath 20. A length D1 is equal to a height of the projection 34 plus an outer diameter of the adapter 30. The length D1 is less than a largest inner diameter D3 of the opening 228 but larger than a smallest inner diameter D2 of the opening 228. As such, the projection 34 is facilitated to mount inside the sheath 20 by such configuration and the flexibility of the plastic sheath 20 in assembling the adapter 30, the plunger 10, and the sheath 20 together. Also, the molding of the sheath 20 can be much simplified. Otherwise, as done in conventional configuration, a different complicated approach is required if the sheath 20 is not formed of plastic. In detail, the length D1 is longer than an inner diameter of the sheath 20. This prevents the projection 34 from mounting inside the sheath 20 directly. Instead, a slit (not shown) should be formed in the rear end portion of the sheath 20. It is required to slide the projection 34 to pass through the slit for mounting the projection 34 inside the sheath 20. Thereafter, join the slit by heating. Finally, a complete syringe is formed. In view of the above, this approach is disadvantageous and the configuration mentioned above of the present invention is greatly advantageous.

For describing the adaptability of the syringe of the invention, second, third, and fourth preferred embodiments will now be discussed in detail below.

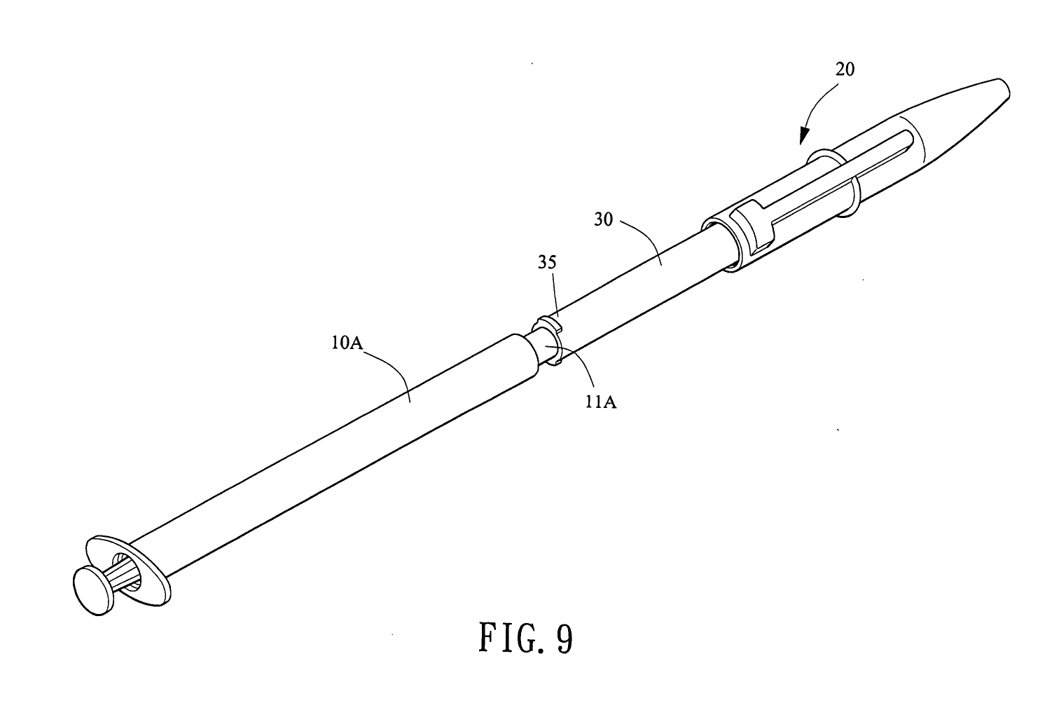

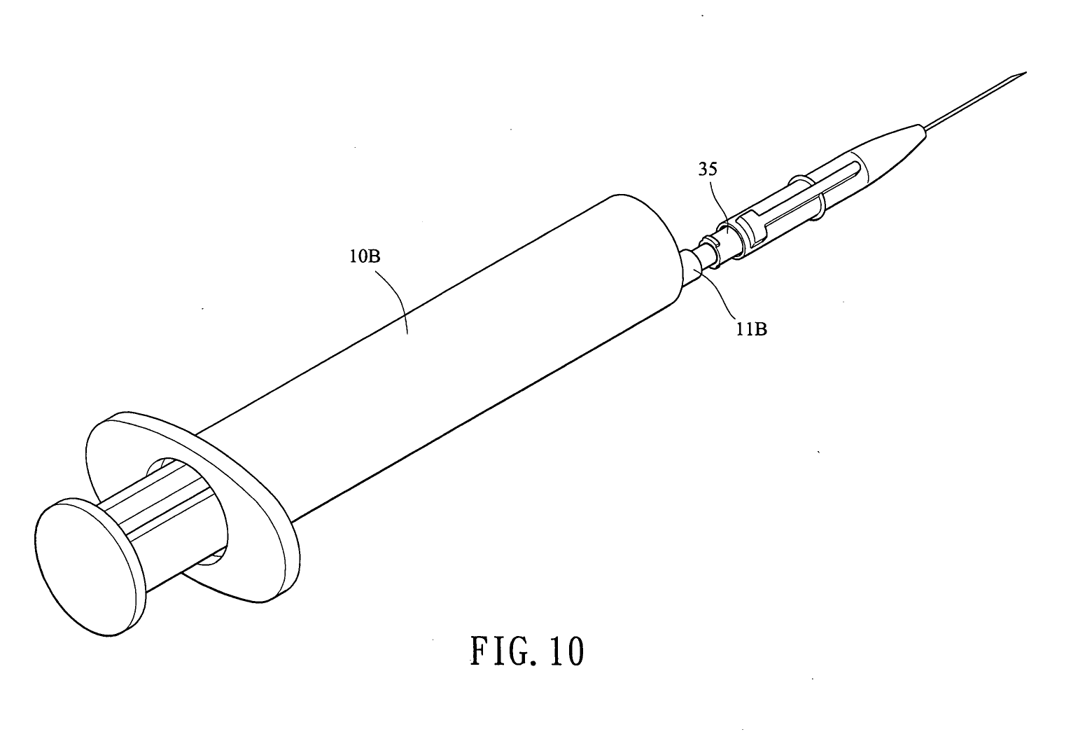

Referring to FIGS. 9 and 10, there is shown an adaptable safety syringe constructed in accordance with second and third preferred embodiments of the invention respectively. The second preferred embodiment substantially has same structure as the first preferred embodiment. The difference between the first and the second preferred embodiments, i.e., the characteristic of the second preferred embodiment is detailed below. A plunger 10A has a relatively small diameter and a forward nose 11A snugly fitted in the rear bore of the first cylindrical section 35 of the adapter 30. Likewise, the third preferred embodiment substantially has same structure as the first preferred embodiment. The difference between the first and the third preferred embodiments, i.e., the characteristic of the third preferred embodiment is detailed below. A plunger 10B has a relatively large diameter and a forward nose 11B snugly fitted in the rear bore of the first cylindrical section 35 of the adapter 30.

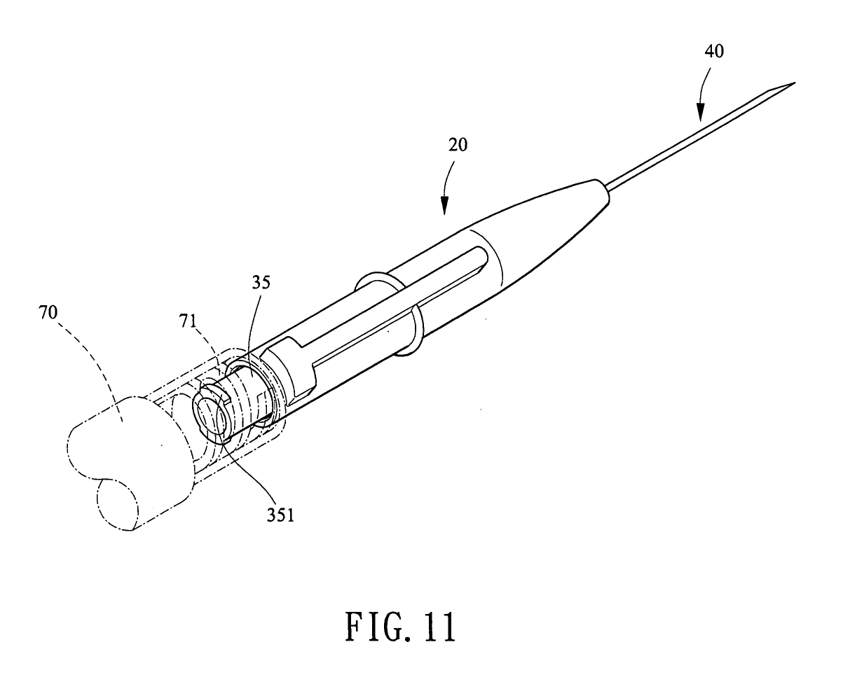

Referring to FIG. 11, there is shown an adaptable safety syringe constructed in accordance with a fourth preferred embodiment of the invention. The fourth preferred embodiment substantially has same structure as the first preferred embodiment. The difference between the first and the fourth preferred embodiments, i.e., the characteristic of the fourth preferred embodiment is detailed below. The flanges 351 of the adapter 30 are threadedly secured to a female adapter 71 of an IV infusion set 70.

Additional benefit of the invention includes: The sheath is adapted to couple to one of a variety of typical syringe plungers or IV infusion sets by means of the adapter without need of any modification or change or variation of the plunger or the IV infusion set, thereby greatly increasing compatibility and adaptability of the invention to the existing syringe plungers or IV infusion sets.

Moreover, the projection 34 can be formed as a cube or polygonal member, the groove 220 can be shaped as an L or I-shaped groove, or the rearward intersection 221R and the recess 224 can be formed as a single element in any of other embodiments.

While the invention herein disclosed has been described by means of specific embodiments, numerous modifications and variations could be made thereto by those skilled in the art without departing from the scope and spirit of the invention set forth in the claims.

1. An adaptable, retractable syringe comprising:
a plunger comprising a forward nose;
a hollow cylindrical adapter snugly fitted on the forward nose, the adapter including a rear first cylindrical section, a forward second cylindrical section having an outer diameter smaller than that of the first cylindrical section, and a projection on a circumference of the first cylindrical section adjacent the second cylindrical section;
a hypodermic needle unit including a needle housing snugly fitted on the second cylindrical section and a needle extended from the needle housing; and
a plastic sheath for enclosing the adapter and the needle unit in a non-operating position, the sheath comprising a lengthwise groove for defining a sliding mode for the projection, the groove having a vertical portion including a forward stop and a rearward intersection and a horizontal portion including a recess with the projection disposed therein in the non-operating position, a lock slot opposite to the recess, and an elongate intermediate cross piece abutted on an entrance portion of the lock slot, and an aperture in a forward end of the sheath,
wherein in a first dispensing fluid, move the projection from the recess to the groove via the horizontal portion and the rearward intersection and push the plunger forward to slide the projection along the sliding mode of the groove and the needle to extend through the aperture until the projection is stopped by the forward stop; responsive to dispensing fluid, slide the projection rearward along the sliding mode of the groove from the forward stop to the recess via the rearward intersection and the horizontal portion; and responsive to again disposing the projection in the recess, clockwise rotate the adapter with respect to the sheath to move the projection from the recess to and lock in the lock slot by passing under the cross piece.

2. The syringe of claim 1, wherein the sheath further comprises a dust cover disposed on the groove.

3. The syringe of claim 1, wherein the groove is shaped as a T.

4. The syringe of claim 1, wherein the groove is shaped as an L.

5. The syringe of claim 1, wherein the projection comprises a first side of D shape, a second side of trapezoidal shape, a third side of D shape, a fourth side of trapezoidal shape, a square top, and a square, flat bottom with an area of the first side larger than that of the second side, an area of the second side larger than that of the third side, an area of the third side larger than that of the fourth side, and the top being arcuate downward from the higher second side to the lower fourth side and from the higher first side to the lower third side for facilitating the projection to slide to lock in the lock slot by passing under the cross piece.

6. The syringe of claim 1, further comprising a slip resistant ring formed on a circumference of the sheath.

7. The syringe of claim 1, further comprising a bell shaped opening and an abutted shoulder formed at a rear end of the sheath wherein the shoulder has an inner diameter smaller than that of the sheath at the rear end of the sheath.

8. The syringe of claim 1, wherein one side of the top of the projection is higher than a top surface of the abutted cross piece for substantially preventing the projection from disengaging the lock slot accidentally.

9. The syringe of claim 1, wherein the adapter further comprises two opposite rear circumferential flanges for threadedly securing to an intravenous infusion device.

10. The syringe of claim 1, wherein the recess of the horizontal portion is formed by a shape of square.

11. The syringe of claim 1, wherein the lock slot opposite to the recess is formed by a shape of square.
